(19) United States
(12) Patent Application Publication
(10) Pub. No.: US 2009/0188499 A1
(43) Pub. Date: Jul. 30, 2009

(54) Method and apparatus for customizing delivery of an oxygen-enriched gas to a user

(76) Inventors: Michael P. Chekal, Brighton, MI (US); Roger Horton, Longmont, CO (US)

Correspondence Address: DELPHI TECHNOLOGIES, INC., M/C 480-410-202, PO BOX 5052, TROY, MI, 48007, US

(21) Appl. No.: 12/011,889

(22) Filed: Jan. 30, 2008

(51) Int. Cl. A61M 16/00

(52) U.S. Cl. 128/203.25

(57) ABSTRACT

A method of customizing delivery of an oxygen-enriched gas to a user includes calibrating an oxygen delivery device such that it is configured to deliver oxygen-enriched gas to the user in an amount specific to the user's predicted need, which need is correlated with the user's then-current activity. An apparatus for achieving this method is also disclosed herein.

### BACKGROUND

The present disclosure relates generally to oxygen delivery and, more particularly, to a method of customizing delivery of an oxygen-enriched gas to a user.

Oxygen delivery devices and/or systems are often used to deliver an oxygen-enriched gas to a user. Some of the oxygen delivery devices are configured to deliver the oxygen-enriched gas to the user at a pre-set, substantially constant flow rate. Other oxygen delivery devices are configured to deliver the oxygen-enriched gas to the user in pulses, where each pulse has a pre-set duration and/or pre-set valve timing based upon a predetermined flow setting. In some instances, the oxygen-enriched gas from any of these devices or systems is also delivered to the user at a pre-set, substantially constant concentration and/or volume, typically over a specific time period. Sometimes, however, the user engages in an activity that requires a different pre-set concentration, delivery timing, and/or volume of the oxygen-enriched gas delivered by the oxygen delivery device.

### SUMMARY

A method of customizing delivery of an oxygen-enriched gas to a user includes user-specific calibration of an oxygen delivery device such that it is configured to deliver the oxygen-enriched gas to the user in an amount specific to the user's predicted need, which need is correlated with the user's then-current activity.

### BRIEF DESCRIPTION OF THE DRAWINGS

Features and advantages of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic diagram of an oxygen delivery device;

FIGS. 2A-2C are semi-schematic depictions of embodiments of the oxygen delivery device;

FIG. 3 is a schematic diagram of a testing protocol used in an embodiment of the method of customizing delivery of an oxygen-enriched gas to a user; and

FIG. 4 is a schematic diagram depicting an embodiment of a method of delivering the oxygen-enriched gas to the user.

### DETAILED DESCRIPTION

Embodiment(s) of the method and apparatus of the present disclosure advantageously deliver a substantially adequate amount of an oxygen-enriched gas to a user when the user engages in a specific activity. This is accomplished by customizing the delivery of the oxygen-enriched gas to the user, where delivery of the gas is specific to the user's predicted need. As used herein, the term “user's predicted need” refers to a previously determined amount of the oxygen-enriched gas that the user needs when the user is engaged in a specific activity (e.g., a then-current activity). The delivery of the oxygen-enriched gas is further advantageously customized to deliver an adequate amount of the oxygen-enriched gas to the user in order to satisfy the user's predicted need for a plurality of different activities, whereby the user may select which activity he/she is or will be engaged in.

Customization of the delivery of the oxygen-enriched gas to the user is accomplished by calibrating an oxygen delivery device such that it is configured to deliver the oxygen-enriched gas to the user in an amount specific to the user's predicted need.

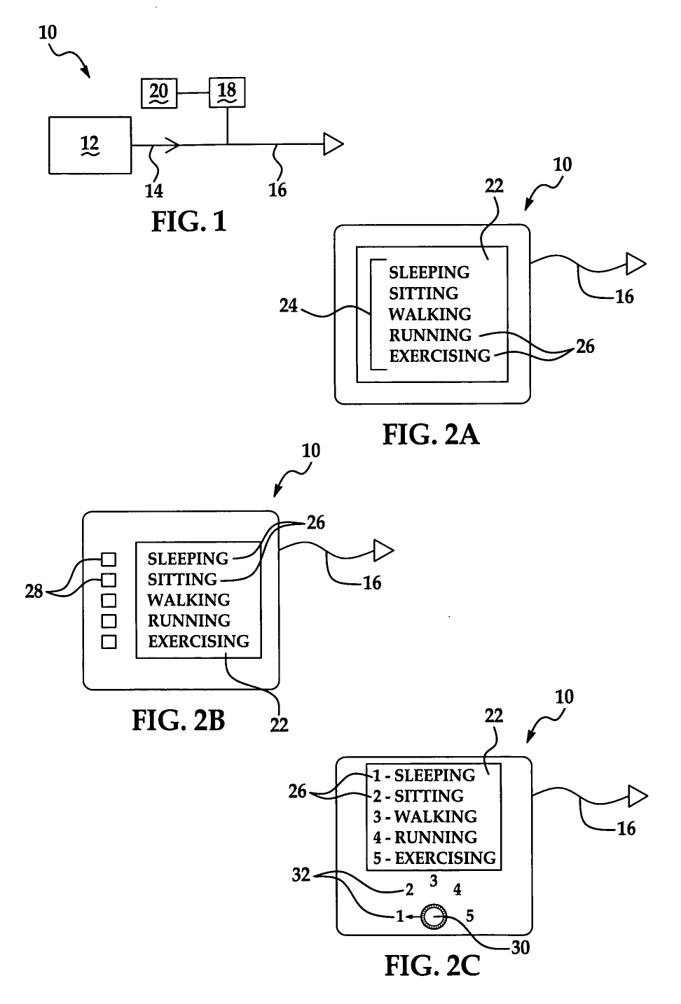

An embodiment of the oxygen delivery device is schematically depicted in FIG. 1. The oxygen delivery device 10 includes an oxygen supply device 12 for supplying the oxygen-enriched gas to the user. In an embodiment, the oxygen supply device 12 is an oxygen tank pre-filled with the oxygen-enriched gas. In another embodiment, the oxygen supply device 12 is an oxygen generation system that generates the oxygen-enriched gas, either on demand based on, e.g., detection of a breath inhalation by the user, or continuously.

The oxygen delivery device 10 may also include a user conduit 14 having a user outlet 16 in fluid communication with the oxygen supply device 12 for delivery of the oxygen-enriched gas to the user. In an example, the user conduit 14 may be formed at least partially from flexible plastic tubing. The user outlet 16 may be any orifice in the user conduit 14 configured to output the oxygen-enriched gas for the user's use. The user outlet 16 may additionally be configured with a nasal cannula, a respiratory mask, or any other suitable device, if desired.

The oxygen delivery device 10 further includes a processor 18. In an embodiment, the processor 18 is operatively connected to the user conduit 14. For continuous-flow oxygen delivery devices, the processor 18 may be configured to control a substantially continuous flow of the oxygen-enriched gas to the user from the oxygen supply device 12. For pulsed-flow oxygen delivery devices, the processor 18 may be configured to control pulses of the oxygen-enriched gas to the user. In a non-limiting example, the pulsed-flow oxygen delivery devices may include a breath detection device (not shown) and the processor 18 may be coupled to the breath detection device. The breath detection device is generally used to detect at least an inhalation of the user, which the processor 18 uses to trigger the delivery of a predetermined volume (i.e., a pulse) of the oxygen-enriched gas to the user. For devices that are configured for both continuous-flow oxygen delivery and pulsed oxygen delivery, the processor 18 may be configured to control both the continuous flow and pulses of the oxygen-enriched gas to the user.

The processor 18 is also configured to receive, process, and store a customized profile of the user. The customized profile is used to set the oxygen delivery device 10 with a plurality of levels, where each level corresponds to the delivery of the adequate amount of oxygen-enriched gas during a then-current activity of the user. As used herein, the term “level” refers to a setting for a predetermined, adequate amount of oxygen-enriched gas to be delivered to the user. The level generally corresponds to a specific activity of the user. In a non-limiting example, the level may be a bolus amount, a change in average flow of the oxygen-enriched gas to the user, an oxygen purity level, a timing between pulses of a predetermined amount of the delivered oxygen-enriched gas, and/or the like, and/or combinations thereof.

The setting or level is programmed into the oxygen delivery device 10 and, as provided above, generally corresponds to a particular activity of the user. Non-limiting examples of activities include sleeping, standing, sitting, walking, running, stair climbing, exercising, and/or the like. It is to be understood that, with any of these activities, the adequate amount, the pressure, the flow rate, and the duration of flow of the oxygen-enriched gas may vary with respect to the intensity of the activity. For example, a user would need a substantially smaller amount of oxygen-enriched gas when he/she is engaged in a light activity such as, e.g., standing, as opposed to when he/she is engaged in a more vigorous activity such as, e.g., walking upstairs. To accommodate these various intensities, sub-levels may also be included for an activity in order to deliver more accurate amounts of the oxygen-enriched gas to the user.

The oxygen delivery device 10 also includes a communications device (not shown) 20 operatively connected to the processor 18. The communications device 20 is configured to receive the customized profile previously created (as will be described further hereinbelow) including the plurality of levels for the delivery of the oxygen-enriched gas to the user. In a non-limiting example, the communications device 20 is a wired cable using any one of a plurality of industry-standard physical interface methods. Non-limiting examples of suitable wired cables include a transistor-transistor logic (TTL) cable, a Recommended Standard 232 (RS232) cable, an RS485 cable, a universal standard bus (USB) cable, a firewire cable, an Ethernet cable, a custom-made cable, and/or the like. In another non-limiting example, the communications device 20 is a wireless communications device for receiving the customized profile from a remote wireless device. The wireless communications device may be selected from those using any one of a plurality of wireless protocols such as, e.g., ZigBee®, Bluetooth®, Wireless Ethernet, a custom-made protocol, and/or the like. In still another non-limiting example, the communications device 20 may be a wired cable (such as those provided above) connected to an external device (e.g., a wired monitor, a personal computer, or the like) configured to send or receive the customized profile from or to the remote device. The communications device 20 is also configured to communicate with a user interface (e.g., a user interface 22 (shown in FIGS. 2A-2C) for inputting the customized profile directly into the oxygen delivery device 10.

The oxygen delivery device 10 yet further includes a means for selecting a level that is set in the device 10. In an embodiment, as shown in FIG. 2A, the means for selecting a level includes a menu 24 of a plurality of levels 26 provided on the user interface 22. The desired level is selected, by the user, by choosing one of the respective levels from the menu 24. Choosing may be accomplished, e.g., by operating a mouse to select the level, touching the level on a touch screen, and/or the like, and/or combinations thereof.

In another embodiment, as shown in FIG. 2B, the means for selecting a level includes a plurality of buttons 28 disposed on the device 10, where each button 28 corresponds to a respective level 26. The buttons 28 are configured to actuate a respective switch, upon depressing the button 26, to cause delivery of the oxygen-enriched gas at the respective level 26.

As shown in FIG. 2B, the buttons 28 are disposed on the device 10 adjacent the user interface 22, where each button 28 lines up with a respective level 26 presented on the user interface 22. Alternatively, the buttons 28 may individually be labeled with suitable alpha numeric indicia such as, e.g., “SLEEPING,” “SITTING,” “1,” “2,” and/or the like, or may individually be labeled with graphical indicia such as, e.g., a picture of a person sleeping, a picture of a person sitting, and/or the like.

In yet another embodiment, as shown in FIG. 2C, the means for selecting a level is a dial 30 operatively connected to the device 10 and configured to selectively move to a plurality of stops 32. Each stop 32 is generally displayed on the device 10 in the form of any suitable alpha numeric or graphical indicia. In the embodiment shown in FIG. 2C, e.g., each stop 32 is labeled as “1,” “2,” “3,” etc. The stops 32 generally correspond to respective levels 26, which are displayed, e.g., on the user interface 22. The stops 32 are configured to actuate a respective switch, upon turning the dial 30 to one of the stops 32, to cause delivery of the oxygen-enriched gas at the respective level.

The embodiments depicted in FIGS. 2A-2C are examples of possible configurations for the means for selecting a level on the oxygen delivery device 10. It is to be understood, however, that other configurations known by one skilled in the art also fall within the spirit and scope of the present disclosure.

As provided above, the method of customizing delivery of the oxygen-enriched gas to the user includes calibrating the oxygen delivery device 10 such that it is configured to deliver the oxygen-enriched gas to the user in an amount specific to the user's predicted need. In an embodiment, the user's predicted need correlates to a default level corresponding to an activity. The oxygen delivery device is set with a plurality of default levels, whereby each default level corresponds to a different activity.

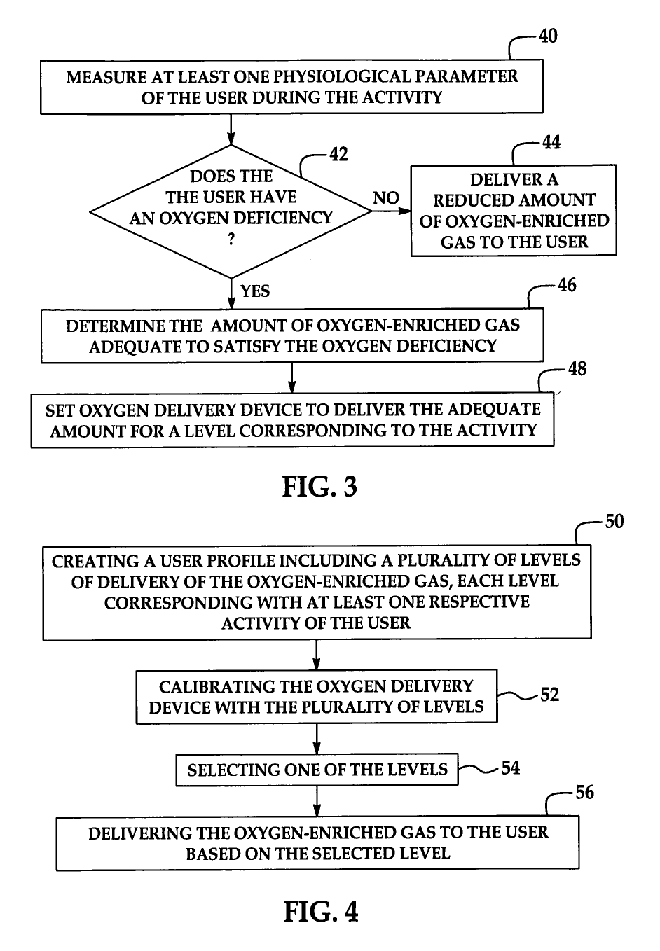

In another embodiment, the user's predicted need correlates to a level corresponding to an activity, where the level is determined via a testing protocol. In this embodiment, once the user's predicted need is determined for the activity, the oxygen delivery device is set with the level, which is correlated to the substantially adequate amount of oxygen-enriched gas to be delivered to the user during the activity. The testing protocol including setting the oxygen delivery device is schematically depicted in FIG. 3.

The testing protocol begins by measuring the value of at least one physiological parameter of the user during a specific activity of the user (as shown by reference numeral 40). In a non-limiting example, blood oxygen saturation of the user are measured by a clinician while the user engages in the activity. Other non-limiting examples of physiological parameters include arterial blood gases, blood pressure, pulse rate, respiration rate, body weight, and/or the like. The measurement(s) are either 1) manually recorded by the clinician, or 2) electronically recorded via an electronic tester or monitor such as, e.g., a pulmonary function tester.

Based on the measurement(s) of the physiological parameter(s), the testing protocol includes determining if the user has an oxygen deficiency (as shown by reference numeral 42). As used herein, the term “oxygen deficiency” refers to the amount of oxygen that a user is lacking, as compared to a clinically acceptable and specific amount for the user when the user is engaged in an activity. The user is generally oxygen deficient when the clinician determines that the user's oxygen saturation level, for example, needs an improvement. In a non-limiting example, the oxygen deficiency is determined by comparing the measured physiological parameter(s) with clinically accepted or predetermined values.

In the event that the measured physiological parameter(s) is/are at or exceed the clinically accepted value(s), the user is considered not to be oxygen deficient for that particular activity. In this case, the oxygen delivery device is calibrated so that the device delivers a reduced amount of oxygen-enriched gas to the user during that particular activity (as shown by reference numeral 44).

However, in the event that the measured physiological parameter(s) is/are below the clinically accepted value(s), the user is considered to be oxygen deficient for that particular activity. In this case, the amount of oxygen-enriched gas adequate to satisfy the oxygen deficiency is then determined (as shown by reference numeral 46). It is to be understood that the amount adequate to satisfy the oxygen deficiency is clinically determined for each user. It is further to be understood that the adequate amount may also be dependent on then-current standard(s) of care adopted by the relevant medical authority. In a non-limiting example, the determined adequate amount ranges from about 1 LPM to about 5 LPM.

In an embodiment, determination of the adequate amount for a particular activity is accomplished via an algorithm. The algorithm generally includes receiving, via the communication device, the measured physiological parameter(s) of the user while the user is engaged in a particular activity. In a non-limiting example, the physiological parameter(s) is/are measured using a vital signs monitor such as, e.g., A VitalPoint™ Home device, manufactured by Delphi Medical Systems, Troy, Mich., which is connected to the user while the user is engaged in the activity. The delivery of the oxygen-enriched gas to the user is then automatically adjusted until the measured physiological parameter(s) fall within a predetermined, accepted range. For example, the vital signs monitor, which is connected to the user during the activity, controls the delivery of the oxygen-enriched gas until the physiological parameter(s) has/have substantially stabilized. Once the delivery of the oxygen-enriched gas has been properly adjusted, the oxygen delivery device is set with the level associated with the particular activity.

Once the adequate amount is determined, the oxygen delivery device 10 is calibrated by setting the device to deliver the adequate amount to the user during the activity when the user selects, on the device 10, the level corresponding to the activity (as shown by reference numeral 48). Setting may be accomplished by manually inputting the adequate amount for the level corresponding to the particular activity directly into the oxygen delivery device. In a non-limiting example, the clinician may manually input the adequate amount into the device via the user interface 22, a keyboard, a personal digital assistant (PDA) connected to the device or, if the device is connected to a network, to the network, and/or other suitable inputting means. In another non-limiting example, the clinician may select an amount corresponding to the adequate amount from a table including a plurality of entries of amounts for other users engaged in the same activity. The entries may be arranged in the table by, e.g., user weight, user age, disease, and/or the like, and/or combinations thereof.

In another embodiment, setting the oxygen delivery device 10 may be realized using a remote wireless device. This may be accomplished by manually inputting the adequate amount for the level corresponding to the particular activity into the remote wireless device and transmitting the adequate amount from the remote device to the oxygen delivery device. Non-limiting examples of suitable remote wireless devices include any device supporting a wireless communication protocol such as, e.g., a PDA or a cellular phone. In another non-limiting example, the remote wireless device may be a personal computer using a communication protocol such as, e.g., ZigBee®, Bluetooth®, wireless Ethernet, and/or the like.

It is to be understood that once the oxygen delivery device 10 is set, the user and/or the clinician may override the settings and manually adjust the oxygen delivery device 10 to deliver a higher or lower amount of oxygen-enriched gas. For example, the oxygen delivery device may be set to deliver a substantially adequate amount of oxygen-enriched gas to the user when the user is engaging in an activity at sea level. However, at higher altitudes, the user may need a higher amount of oxygen-enriched gas to perform the same activity. In this case, the user may manually adjust the oxygen delivery device 10 to deliver an additional amount of the oxygen-enriched gas. The device 10 may also include components configured to, and be programmed to detect, what altitude (typically based on barometric pressure readings) it is operating in, and adjust its settings automatically.

In a non-limiting example, the steps of the testing protocol may be applied by the clinician manually. In another non-limiting example, the steps of the testing protocol may be programmed into a handheld device, and the handheld device may then prompt the clinician, visually or verbally, through the steps of the testing protocol. Non-limiting examples of handheld devices include PDAs, cellular phones, palmtop computers, laptop computers, and/or the like. It is to be understood that a user, in addition to or in place of the clinician, may complete the steps of the testing protocol. In this case, the user would then set the oxygen delivery device 10 with the appropriate levels.

It is to be understood that, although the user may experience an oxygen deficiency for each activity that the user engages in, the substantially adequate amount to satisfy the oxygen deficiency may vary from one activity to another. Accordingly, the testing protocol is applied for each activity that the user may ultimately engage in. For example, if the user walks, runs, climbs stairs, sleeps, and sits, the testing protocol will be applied for each of these activities. A user profile, as described above, including the five activities (i.e., walking, running, climbing stairs, sleeping, and sitting) is created from the results of the testing protocol. The oxygen delivery device 10 is then set with the user profile so that the device 10 is capable of delivering the adequate amount, if any, for the five different activities. It is to be understood that a single button 28, stop 32 and/or the like may be associated with one or more activities. For example, stop 32 “1” (which can also be associated with about 1 liter per minute, if desired) may have associated therewith both “sleeping” and “sitting.” Similarly, stop 32 “2” may have associated therewith both “standing” and “walking at 1 mph,” and so on.

In another embodiment, the oxygen delivery device 10 is calibrated such that a level is correlated with a time of day. For example, if the user takes a nap every day from 1:00 p.m. to 2:00 p.m., the oxygen delivery device 10 will automatically deliver the adequate amount of oxygen-enriched gas (if any) to the user at 1:00 p.m. every day. The adequate amount delivered corresponds with the level associated with the activity of sleeping. Correlating the activity with the time of day may be accomplished by setting the device with a starting time (e.g., 1:00 p.m.) and a stopping time (e.g., 2:00 p.m.) for a particular activity. Correlating the activity with the time of day may also be accomplished by selecting the activity (e.g., sleeping) and setting the device with the starting time and stopping time for that activity. In either case, the device may automatically change the delivery of the oxygen-enriched gas to the user based on the time of day and the activity that the user is or will be engaged in.

The time of day may also be tracked using, e.g., a real time clock operatively connected to the oxygen delivery device 10, where the real time clock is set to the local time of the user. The oxygen delivery device 10 may further be configured to alert the user when the delivery of the oxygen-enriched gas changes at the selected starting time. The alert allows the user to either accept or reject the delivery change. If the user rejects the deliver change, the user may then have the option to override the setting and select a new setting.

In a further embodiment, the delivery device 10 may also be configured to be set back to its default settings after a predetermined period of time, after predefined events take place, when a usage pattern is detected, or combinations thereof. This may be accomplished via the real time clock or via an internally programmed calculation or algorithm. Without being bound to any theory, it is believed that resetting the delivery device 10 will encourage the user and/or clinician to periodically check the device 10 to make sure that the device is properly calibrated and/or is delivering the correct amount of oxygen-enriched gas to the user.

In yet a further embodiment, the oxygen delivery device 10 is calibrated such that a level is correlated with a treatment protocol, a physiology of the user, a disease, a stage of a disease, or combinations thereof. For example, if the user suffers from an upper respiratory disease, the oxygen delivery device 10 may be set to deliver an adequate amount of oxygen-enriched gas based on the needs of the user while engaged in an activity and while the user is plagued with the respiratory disease. Correlating the delivery of the oxygen-enriched gas with the treatment protocol, the physiology of the user, the disease, the stage of a disease, or combinations thereof may be accomplished via an algorithm. The algorithm generally includes correlating standardized settings for each activity based on at least one parameter provided by the user or the clinician. The parameter(s) include, but are not limited to, a disease, a disease state, a weight of the user, a lung capacity of the user, and/or the like. Correlating may be accomplished using a clinically-accepted formula or a look-up table.

Also disclosed herein is a method of delivering an oxygen-enriched gas to the user. With reference to FIG. 4, the method includes creating a user profile including a plurality of levels of delivery of the oxygen-enriched gas, each level corresponding with at least one respective activity of the user (as shown by reference numeral 50); calibrating the oxygen delivery device with the plurality of levels (as shown by reference numeral 52); selecting one of the respective activities (as shown by reference numeral 54); and delivering the oxygen-enriched gas to the user based on the selected activity (as shown by reference numeral 56).

It is to be understood that the term “connect/connected” or the like is broadly defined herein to encompass a variety of divergent connecting arrangements and assembly techniques. These arrangements and techniques include, but are not limited to (1) the direct connection between one component and another component with no intervening components therebetween; and (2) the connection of one component and another component with one or more components therebetween, provided that the one component being “connected to” the other component is somehow operatively coupled to the other component (notwithstanding the presence of one or more additional components therebetween).

While several embodiments have been described in detail, it will be apparent to those skilled in the art that the disclosed embodiments may be modified. Therefore, the foregoing description is to be considered exemplary rather than limiting.

What is claimed is:

1. A method of customizing delivery of an oxygen-enriched gas to a user, the method comprising:
calibrating an oxygen delivery device such that it is configured to deliver the oxygen-enriched gas to the user in an amount specific to the user's predicted need, which need is correlated with the user's then-current activity.

2. The method as defined in claim 1 wherein the user's predicted need correlates to default levels corresponding to at least one activity, and wherein the method further comprises setting the oxygen delivery device with the default levels.

3. The method as defined in claim 1 wherein the user's predicted need correlates to a level determined via a testing protocol, the testing protocol including:
measuring a value of at least one physiological parameter of the user during at least one activity;
determining, from the measurement, if the user has an oxygen deficiency; and
determining, if the user does have an oxygen deficiency, the amount of oxygen-enriched gas adequate to substantially satisfy the oxygen deficiency;
wherein the level corresponds to the at least one activity.

4. The method as defined in claim 3 wherein the adequate amount substantially ranges from about 1 LPM to about 5 LPM.

5. The method as defined in claim 3 wherein the amount of oxygen-enriched gas adequate to substantially satisfy the oxygen deficiency is determined by:
receiving, via the communication device, the measured at least one physiological parameter of the user while the user is engaged in the at least one activity;
automatically adjusting the oxygen delivery device until the at least one measured at least one physiological parameter falls within a predetermined range; and
setting the oxygen delivery device with the level associated with the at least one activity.

6. The method as defined in claim 3 wherein calibrating the oxygen delivery device includes setting the oxygen delivery device to deliver the adequate amount determined from the testing protocol for the level corresponding to the at least one activity.

7. The method as defined in claim 6 wherein the setting is accomplished by:
manually inputting the level corresponding to the at least one activity directly into the oxygen delivery device; or
manually inputting the level corresponding to the at least one activity into a remote wireless device and transmitting the level from the remote wireless device to the oxygen delivery device.

8. The method as defined in claim 3 wherein calibrating the delivery device includes:
prompting the user through the testing protocol for the at least one activity via a handheld device;
inputting the determined adequate amount of oxygen-enriched gas into the oxygen delivery device; and
setting the oxygen delivery device to deliver the determined adequate amount for the level corresponding to the at least one activity.

9. The method as defined in claim 3 wherein the oxygen deficiency is determined by comparing the measured value of the at least one physiological parameter of the user with a predetermined value.

10. The method as defined in claim 3, further comprising:
correlating the at least one activity with a time of day; and
setting the oxygen delivery device to deliver the determined adequate amount for the level corresponding to the at least one activity, and corresponding to the then-current time of day.

11. The method as defined in claim 3, further comprising:
correlating the delivery of the determined adequate amount of oxygen-enriched gas with a treatment protocol, a physiology of the user, a disease, a stage of a disease, or combinations thereof; and
setting the oxygen delivery device to deliver the determined adequate amount for the level corresponding to the at least one activity, and corresponding to the delivery correlation.

12. The method as defined in claim 2 wherein the at least one activity is selected from sitting, sleeping, standing, walking, running, driving, stair climbing, exercising, and combinations thereof.

13. A method of delivering an oxygen-enriched gas to a user, the method comprising:
creating a user profile including a plurality of levels of delivery of the oxygen-enriched gas, each level corresponding with at least one respective activity of the user;
calibrating the oxygen delivery device with the plurality of levels;
selecting one of the levels; and
delivering the oxygen-enriched gas to the user based on the selected level.

14. The method as defined in claim 13 wherein the selecting is accomplished by choosing the one of the levels from a menu of levels, wherein the menu is provided on a graphical user interface operatively connected to the oxygen delivery device.

15. The method as defined in claim 14 wherein the choosing is accomplished by:
depressing one of a plurality of buttons present on the oxygen delivery device, each button corresponding to at least one of the levels and configured to actuate a respective switch to cause delivery of the oxygen-enriched gas at a respective one of the plurality of levels; or
turning a dial to one of a plurality of stops, each stop corresponding to at least one of the levels and configured to actuate a respective switch to cause delivery of the oxygen-enriched gas at a respective one of the plurality of levels.

16. The method as defined in claim 13 wherein each level of the plurality of levels is determined via a testing protocol, the testing protocol including:
measuring at least one of blood oxygen saturation or arterial blood gasses of the user during at least two activities;
determining, from the measurement, if the user has an oxygen deficiency; and
determining, if the user does have an oxygen deficiency, the amount of oxygen-enriched gas adequate to substantially satisfy the oxygen deficiency;
wherein the level corresponds to the at least one activity.

17. The method as defined in claim 16 wherein calibrating the oxygen delivery device includes setting the oxygen delivery device to deliver the adequate amount of oxygen-enriched gas determined from the testing protocol for a level from the plurality of levels corresponding to the selected one of the respective activities.

18. The method as defined in claim 17, further comprising manually adjusting the oxygen delivery device to deliver an other amount of oxygen-enriched gas after the oxygen delivery device has been set.

19. The method as defined in claim 13 wherein each of the plurality of levels for the delivery of the oxygen-enriched gas is selected from a bolus amount, a change in average flow, an oxygen purity level, a timing between pulses of a predetermined amount of the delivered oxygen-enriched gas, and combinations thereof.

20. An oxygen delivery device, comprising:
a communications device configured to receive a customized profile for a user including a plurality of levels for the delivery of oxygen-enriched gas to the user, each level corresponding with a different activity of the user;
a processor for processing the customized profile, thereby setting the device such that a specific level is correlated with at least one respective activity;
means for selecting a level; and
means for delivering the oxygen-enriched gas to the user based on the selected level.

21. The oxygen delivery device as defined in claim 20 wherein the communications device may be a wired cable that communicates with a user interface for inputting the customized profile directly into the device, a wired external device for receiving the customized profile from a remote wireless device, or a wireless communications device for receiving the customized profile from the remote wireless device.

22. The oxygen delivery device as defined in claim 20 wherein the means for selecting the activity is a menu of a plurality of activities provided on a user interface operatively connected to the oxygen delivery device.

23. The oxygen delivery device as defined in claim 20 wherein the means for selecting the activity comprises:
a plurality of buttons disposed on the oxygen delivery device, each button corresponding to at least one of the levels and configured to actuate a respective switch to cause delivery of the oxygen-enriched gas at a respective one of the plurality of levels; or
a dial configured to selectively move to a plurality of stops, each stop corresponding to at least one of the levels and configured to actuate a respective switch to cause delivery of the oxygen-enriched gas at a respective one of the plurality of levels.

24. The oxygen delivery device as defined in claim 20 wherein the oxygen delivery device is configured to automatically change the delivery of the oxygen-enriched gas to the user based on a particular time of day, a treatment protocol, a physiology of the user, a disease, a stage of a disease, or combinations thereof.
